(19) United States
(12) Patent Application Publication
(10) Pub. No.: US 2024/0390637 A1
(43) Pub. Date: Nov. 28, 2024

(54) Control Device and Method for Assisting a Multi-Phase Breathing Exercise of a Passenger of a Vehicle

(71) Applicant: Bayerische Motoren Werke Aktiengesellschaft, Muenchen (DE)

(72) Inventor: Etienne Iliffe-Moon, Menlo Park, CA (US)

(21) Appl. No.: 18/646,549

(22) Filed: Apr. 25, 2024

(30) Foreign Application Priority Data
May 26, 2023 (DE) 10 2023 113 862.6

(51) Int. Cl. A61M 21/02; A61M 21/00; B60Q 9/00; G16H 40/67

(52) U.S. Cl. CPC A61M 21/02; B60Q 9/00; G16H 40/67; A61M 2021/0088; A61M 2205/583; A61M 2230/40; A61M 2230/63

(57) ABSTRACT

A control device for assisting a multi-phase breathing exercise of a passenger of a vehicle is provided. The control device is configured to control an illustration device to illustrate instruction steps of the multi-phase breathing exercise to the passenger, —monitor breathing actions and/or states of the passenger while the instructions are being illustrated, evaluate a correlation of the monitored breathing actions and/or states to the illustrated instruction steps, and provide a feedback to the passenger based on the evaluated correlation.

This application claims priority of German patent application no. 10 2023 113 862.6 filed on May 26, 2023, which is incorporated herein by reference in its entirety.

### TECHNICAL FIELD

The present disclosure relates to a control device for assisting a multi-phase breathing exercise of a passenger of a vehicle, a system comprising the control device, a vehicle comprising the control device or the system, and/or a method for assisting a multi-phase breathing exercise of a passenger of a vehicle. Additionally, or alternatively, the present disclosure relates to a computer program and/or a computer-readable medium comprising instructions which, when executed by a computer, cause the computer to at least partially carry out the method.

### BACKGROUND

Respiration is a unique function of the body as it is an autonomous and unconscious function that can also be consciously controlled. Breathing also serves other functions, acting as a mechanism for speech, laughter and other expressions of emotion, it also supports reflexes such as yawning, coughing and sneezing.

Beyond sustaining life, breathing can most significantly alter the human psycho-physiological state. Controlled breathing has been shown to alter both autonomic and central nervous systems activities as well as the psychological state in the subjects of research studies.

Mindfulness and controlled breathing exercises have long been associated together, in eastern medicine, meditation and yoga.

Whilst there are many gaps in our scientific knowledge about how the mind and body work, in recent times much academic and scientific research has shown the there are many benefits of controlled breathing and attempted to explain the physiological and psychological basis of the effect.

Research studies have shown controlled breathing can for example result in increased comfort, relaxation, pleasantness, vigor and alertness, and reduced symptoms of arousal, anxiety, depression, anger, and confusion.

Mindful breathing offers proven wellness and well-being benefits.

There is a need for a mindful breathing experience that can enable a user to effectively control physiological processes (e.g., respiration rate, heart rate, oxygen levels, etc.) and also influence the psycho-physiological state of the user to reduce arousal, stress and anxiety and improve relaxation, focus, attention and holistically center the mind and the body.

There are applications that promote breathing exercises, but these conventionally ask users to breathe in and out as instructed by the GUI (e.g., the user breathes in rhythm or in synchronization with the GUI. However, these apps are one-directional and do not monitor the state, activity or behavior of the user—there is no feedback loop into the system. A feedback loop from the system presents opportunities to respond to the performance of the user, to modify the experience accordingly and additionally create a more holistic experience by incorporating aspects of the vehicle systems/sub-systems to create a holistic multi-sensory experience.

There is a need for an intelligent and immersive multi-sensory experience that provides wellness and wellbeing benefits within the vehicle cabin.

European patent publication no 3537451A1 describes a system for improving posture and deep breathing, comprising a sensor device, a posture/breathing improvement software program, comprising one, both, or a combination of a posture improvement system interface and a breathing improvement system interface, and one or more user devices. The sensor device is physically associated with a user, communicates with the posture improvement software program, and comprises one or more sensors for monitoring positions and movements of the user. The system calculates one or more optimum postural positions and breathing exercises for the user, based on data communicated by the sensor device and collected information about the user. The system monitors a conformance of the user with the optimum postural positions and displays the conformance on the posture improvement system interface. The system detects and notifies the user of one or more non-conformances, such that a user is reminded to maintain at least one optimum postural position and periodically take deep breaths.

U.S. patent publication no. 2019/061772 A1 describes a system for monitoring the state of health of a vehicle occupant, comprising a control unit, which comprises the following: a receiver for the wireless reception of physiological parameters of at least one unit which is worn on the body, which comprises one or more sensors for determining one or more physiological parameters of the vehicle occupant, and a diagnostic module which is designed to derive information regarding the state of health, the state of well-being, or of illnesses, at least partially on the basis of the physiological parameters received. The control unit is also designed to provide the vehicle occupants with information regarding the state of health by means of at least one output unit of the vehicle and to initiate at least one of the following steps: adapt vehicle functions to the state, or, by means of at least one output unit of the vehicle, to suggest or interactively to carry out measures that improve the state.

In view of this prior art, the object of the present disclosure is to disclose a device and/or a method, each of which is suitable for enriching the prior art.

### SUMMARY

The object is solved by the features of at least some embodiments and implementations of the disclosure.

Accordingly, the object is solved by a control device for assisting a multi-phase breathing exercise of a passenger of a vehicle.

The control device is configured to control an illustration device (e.g., a display) to illustrate (e.g., to display) instruction steps of the multi-phase breathing exercise to the passenger.

The control device is configured to monitor breathing actions and/or states of the passenger while the instructions are being illustrated.

The control device is configured to evaluate a correlation of the monitored breathing actions and/or states to the illustrated instruction steps.

The control device is configured to provide a feedback to the passenger based on the evaluated correlation.

The control device may be configured for electronic data processing and/or at least partially configured as an electronic data processing device, which may have, e.g., one or more microprocessors and data storages. The control device may, e.g., include software and/or at least one algorithm for processing and evaluating sensor signals, data and/or images for monitoring the breathing actions and/or states and evaluating the correlation.

The control device or control unit may be part of or represent a driving assistance system. The control device may be, e.g., an electronic control unit (ECU). The electronic control unit may be an intelligent processor-controlled unit which can communicate with other modules, e.g., via a central gateway (CGW), and which, if necessary, may form (part of) the vehicle electrical system via field buses such as the CAN bus, LIN bus, MOST bus, FlexRay and/or via the automotive Ethernet, e.g., together with telematics control units and/or an environment sensor system.

It is possible that the control device controls functions relevant to the driving behavior of the (motor) vehicle, such as the steering, the engine control, the power transmission, and/or the braking system. In addition, driver assistance systems such as a parking assistant, adaptive cruise control (ACC), lane departure warning, lane change assistant, traffic sign recognition, light signal recognition, approach assistant, night vision assistant, and/or intersection assistant may be controlled by the control device.

The control device described above provides the advantage of, among other things, an improved concept of a breathing guidance and breathing (or respiration) determination which are combined to create an interactive breathing experience. The experience immerses the passenger in a multi-sensory experience that, e.g., gamifies and monitors the passenger's behavior and performance.

Possible further implementations of the control device described herein are explained in detail below.

The multi-phase breathing exercise may be composed of multiple phases comprising at least an inhale phase and at least an exhale phase and optionally at least a breathing hold phase. The illustrated instruction steps may specify a breathing duration, a breathing depth and/or a breathing rate for each of the multiple phases.

The multiple phases may be repeated one or more times. The multiple phases may comprise two, three, four or more phases.

The multiple phases may be symmetrical, wherein each of the multiple phases may have the same duration and/or breathing depth.

The multiple phases may be asymmetrical, wherein at least two of the multiple phases may have the different durations and/or different breathing depths.

The control device may be configured to evaluate the correlation by tracking whether the multiple phases are performed by the passenger in accordance with the illustrated instruction steps. In other word, the control device may be configured to evaluate the correlation by comparing the monitored breathing actions and/or states of the passenger with the illustrated instruction steps.

The instruction steps may comprise additional instructions regarding body gestures. Each of the body gestures may a facial and/or hand gesture providing a measure of one of the breathing actions and/or states.

The body gestures may comprise a body shape, motion and/or activity. The body gestures may comprise a mouth shape, an eye opening, an eye closure and/or a chest inflation.

A hand gesture may be both hands of the passenger forming a cradle (e.g., hand-in-hand), finger tips of both hands touching to form a bridge, and/or a thumb and finger at each hand touching (e.g., forming a namaste sign). A hand gesture may be at least a finger on the head and/or face (e.g., nose) of the passenger.

A facial gesture may comprise a gesture involving the eyes, mouth, nose and/or ears of the passenger. A facial gesture may be open eyes, closed eyes and/or blinking. A facial gesture may be a smile, a closed mouth, and/or an open mouth (e.g., for blowing movement or a sigh). A facial gesture may be a right finger positioned next to the right nostril and/or a left finger positioned next to the left nostril. A facial gesture may be fingers closing the ears.

The states may be (or may comprise) breathing states of the passenger. The states may comprise heart rate states (e.g., stress and/or relaxation) of the passenger.

The control device may be configured to monitor the breathing actions and/or states by receiving, from a sensor device, (e.g., real-time) data of the passenger, such as body movements (and/or heart rate information) of the passenger. The control device may be configured to fuse the received data, e.g., if the data comprises multiple data types (from multiple sensor types of the sensor device).

The control device may be configured to monitor the states by receiving, from the sensor device, heart rate information (e.g., a pulse, a blood volume pulse (BVP), a heart rate variability (HRV), etc.) of the passenger. The sensor device may comprise an electrocardiogram (ECG) sensor for monitoring heart rate information. Advantageously, the heart rate information allows to evaluate the state of stress and/or relaxation of the passenger before, during and after the exercises. The heart rate information may be part of the gamification of the experience and/or measure the overall success of the experience and past history and/or performance.

The control device may be configured to monitor the breathing actions and/or states by receiving, from a sensor device, (e.g., real-time) data regarding recordings of body gestures of the passenger, and identifying the body gestures in (and/or based on) the received data. A mouth shape may be (e.g., directly) associated with an inhale or an exhale (of the passenger).

The control device may be configured to monitor the breathing actions and/or states by receiving, from a sensor device, data regarding recordings of at least one chest and/or belly expansion and at least one chest and/or belly contraction of the passenger, and determining the at least one chest and/or belly expansion and the at least one chest and/or belly contraction in (and/or based on) the received data.

The illustrated instruction steps may comprise instructions to the passenger to place one of the passenger's hands on the passenger's chest and another of the passenger's hands on the passenger's belly. Determining the at least one chest and/or belly expansion and the at least one chest and/or belly contraction in (and/or based on) the received data may comprise identifying and tracking the passenger's hands as markers of the chest and the belly in (and/or based on) the received data.

Alternatively, markings may be provided on a seatbelt of a vehicle seat on which the passenger is sitting, wherein the markings may mark the passenger's chest and the passenger's belly, respectively, when the seat belt is fastened. Determining the at least one chest and/or belly expansion and the at least one chest and/or belly contraction in (and/or based on) the received data may comprise identifying and tracking the markings in (and/or based on) the received data.

The control device may be configured to provide the feedback by controlling the illustration device to illustrate the evaluated correlation and/or the monitored breathing actions and/or states simultaneously to the instruction steps in a single representation.

Alternatively, or additionally, the control device may be configured to provide the feedback in a visual, audible, haptic and/or olfactory format.

The control device may be configured to determine a performance of the passenger based on the evaluated correlation. The determined performance may comprise a score based on the evaluated correlation, e.g., wherein the score may be higher with higher evaluated correlation. The feedback may comprise the determined performance and/or score.

The control device may be configured to receive identification data to identify the passenger. The passenger may be identified by, e.g., providing identification and/or facial recognition via the illustration device (e.g., a display which may be a touchscreen), the sensor device and/or a digital key.

The control device may be configured to store the evaluated correlation, the monitored breathing actions and/or state, and/or results (such as the determined performance of the passenger) based on the evaluated correlation in a passenger profile of the identified passenger. The passenger profile may provide a track record of the passenger's performance over multiple breathing exercises and multiple vehicle journeys. For instance, the track record may comprise scores of multiple vehicle journeys which may be merged, e.g., summed up, to indicate an overall score.

The control device may be configured to adapt the multi-phase breathing exercise based on passenger performances in previous breathing exercises, a current journey length, a location in the current journey, a mood of the passenger, and/or a state of the passenger. The control device may be configured to adapt the multi-phase breathing exercise using a machine learning and/or artificial intelligence model.

The control device may be configured to determine a mood and/or a state of the passenger, e.g., based on the received data.

The control device may be configured to control the illustration device to illustrate a graphical user interface (GUI) with at least a window illustrating the instruction steps (and optionally the evaluated correlation and/or the monitored breathing actions and/or states simultaneously to the instruction steps).

The control device may be configured to control the illustration device to illustrate the instruction steps by providing visual instructions alongside verbal instructions of the instruction steps.

The control device may be configured to control the illustration device to illustrate the instruction steps (and/or the GUI) in conjunction with an intelligent personal assistant (IPA) for initiating, guiding and/encouraging the passenger during the multi-phase breathing exercise. The GUI may include an IPA avatar for providing visual instructions alongside verbal instructions of the instruction steps.

The control device may be configured to provide an atmosphere inside the vehicle (e.g., In the cabin of the vehicle) in accordance with the multi-phase breathing exercise (and while the instruction steps are illustrated). The atmosphere may comprise a visual, audio, haptic, tactile and/or scent aspect.

The control device may be configured to control an interior lighting device (and/or interior lights) of the vehicle for providing the visual aspect of the atmosphere, e.g., by dimming light inside the cabin of the vehicle. The control device may be configured to control an infotainment system and/or at least a speaker of the vehicle for providing the audio aspect of the atmosphere, e.g., by providing sound effects and/or ambient music.

The control device may be configured to control a ventilation system and/or an air conditioning system of the vehicle for providing the tactile aspect of the atmosphere, e.g., by cooling an airflow and/or a temperature inside the cabin of the vehicle and/or by providing an increased airflow of fresh air inside the cabin.

The control device may be configured to control a seat massage device arranged inside a vehicle seat (e.g., inside a backrest of the vehicle seat) vehicle for providing the haptic aspect of the atmosphere, e.g., by activating and/or changing a massage level of the seat massage device. The massage level may comprise and/or define characteristics of the haptic aspect (an/or the seat massage feature) and optionally other seat related characteristics (e.g., position, orientation, cooling/heating, body support, cushioning (e.g., firm vs. soft)).

The control device may be configured to control a sent device (e.g., a fragrance diffuser) arranged inside the cabin of the vehicle for providing the scent aspect of the atmosphere, e.g., by providing a scent.

The control device may be configured to control the illustration device to illustrate the instruction steps by providing multi-modal instructions of the instruction steps. The multi-modal instructions may include visual, audible, haptic and/or scented instructions.

The foregoing may be summarized in other words and with respect to a possible more specific implementations of the disclosure as described below, wherein the following description is to be construed as not being limiting to the disclosure.

According to the present disclosure, breathing guidance and respiration measurement may be combined to create an interactive breathing experience. The experience may immerse the user in a multi-sensory experience that gamifies and monitors the user's behavior and performance.

The experience may guide users, such as the passenger through multiple breathing exercises sequentially. The breathing exercises may be intended to be multi-phase (2-phase, 3-phase, 4-phase, etc.) and symmetrical and/or asymmetrical.

Additionally, the experience may provide guidance between the exercises, e.g., “free-time”, to breath naturally and at the passenger's own will (e.g., not following an actual exercise), to play with ones breathing pace and rhythm, etc., The next exercise may be adapted according to the passenger behavior during the “free-time” (i.e., the non-guided period).

The system may be intended to recognize/identify users, such as the passenger, and track their performance over multiple journeys. Breathing exercises may be considered as active exercise that is both physical and mental, wherein it may take practice and may be a skill that can be developed over time. The system may be to help the users develop this skill through repetition of multi-phase breathing.

Users may be incentivized to routinely participate in the experience and make experience recommendations based on the experience. Using a gamified approach to measure performance, during an experience/journey and across multiple journeys.

The present disclosure may comprise multiple areas: a GUI, respiration sensing approach, multi-sensory experience (integration within the cabin environment) and user participation (user interaction) and gamification (across a single journey and multiple journeys)

A GUI may be displayed in the vehicle (e.g., via a display/touchscreen (e.g., a center information display, CID), augmented (AR) and/or virtual reality (VR) glasses, a head-up display (HUD), etc.) that may provide guidance on the type of breathing exercise (e.g., multi-phase, asymmetric, chest, diaphragmatic, etc.) and the user breathing rhythm (e.g., breathing rate, phase (e.g., inhale, hold, exhale, hold), duration, depth, etc.).

The GUI may work in conjunction with an intelligent personal assistant (IPA) to initiate, guide and encourage the user. Additionally, the GUI may include an IPA avatar that provides visual instructions alongside verbal instructions.

An illustrated breathing phase model may be based on a 2-dimensional (2D) plane/space or 3-dimensional space (virtual or simulated). For example, the 2D approach may lend itself (preferable) to a touchscreen/display GUI and the 3D may be more suited (preferable) to an AR or VR experience. However, either GUI approach could be adapted for the display output format.

The experience may use a number of approaches to solve the sensor noise problem and additionally solve the user experience engagement problem.

According to one exemplary approach, the guidance may instruct the user to place their left hand on their chest and right hand on the belly. This may help engage and focus the user, but importantly may provide a visual state (i.e., visual cue, event class) for the computer vision approach to identify the hands as a visual marker that can be tracked. The system may track the hand position/motion to directly determine the chest and belly expansion and contraction that directly correlates with breathing phase.

According to another exemplary approach, the guidance may instruct the user to use body gesture (e.g., shape, motion, activity, etc., such as mouth shape, eye opening/closure, chest inflation) to provide an intuitive measure of the breathing action or state. Mouth shape may be directly associated with inhaling and exhaling, wherein there may be an intuitive connection that can be established through the guided experience. This may present a technical benefit such that mouth shape/opening (and potentially other aspects such as eye opening/closure, hand shape/motion, etc.) may be directly measured using the camera (e.g., combined with machine learning or artificial intelligence) and correlated to the GUI instructions/guidance to provide bio-feedback and ultimately assess the performance of the user.

Additionally, additional camera sensing approaches may be incorporated. Respiration may be evaluated by measuring movement of the passenger's clothing features using the camera (e.g., measuring camera pixel movement of clothing features, e.g., shirt/sweater graphics, buttons, pockets, seams, etc.).

The gamification of the experience may be an important aspect to the users' immersion, motivation and adoption of the experience. The bio-feedback may naturally provide a gamification to the experience. Additionally, multi-sensory feedback and scoring may be provided in a visual (e.g., numerical, graphical, etc.), audible, haptic, olfactory, etc. format. This feedback or scoring may be provided during the journey or across multiple journeys (e.g., historical). Multi-sensory aspects may include visual (e.g., GUI, lighting, etc.), sound (e.g., sound effects, ambient music, etc.), haptic/tactile (e.g., seat massage, climatization airflow, temperature, etc.), scent, etc. The experience may also incorporate aspects of other activities, such as meditation, relaxation (e.g., with eye closure), physical exercise, yoga, infotainment (e.g., listening to a podcast, radio, movie) etc.

The system may leverage at least an interior camera and/or sensor fusion approaches with data from other sensors (e.g., inertial measurement unit (IMU), force/strain gauge, radar, microphone, electrocardiogram (ECG) sensor, electroencephalography (EEG) sensor, etc.) may be assessed relative to the guidance provided by the GUI instructions. Additionally, combining camera data with other sensors may provide a more robust data set (that may filter out noise) and feedback loop to the system. Some of these sensors may remotely or directly measure the physical motion or activity of the user (as opposed to measuring a biometric that has a relationship to breathing (e.g., interpreting average respiration rate from ECG data)).

Additionally, these sensors may provide other forms of data on the behavior and state of the user, e.g., emotional state (camera-based emotion detection), heart rate (e.g., from camera data), focus/distraction, etc.

Tests have shown that providing guidance instructing the user to make specific or exaggerated gestures/behaviors in sync with their breathing is easier to accurately monitor and detect in real-time. It helps to minimize the effect of sensor noise or sensor ambiguity to the feedback loop, engages the user in a positive way and smooths out the overall experience. This approach may provide a solution of embedding the solution within the overall experience, which may be made more immersive, entertaining and interesting for the user.

The system may provide guidance and measurement of the user respiration activity (e.g., respiration rate, phase (e.g., inhale-pause-exhale-pause cycle), duration, depth, etc.) during voluntary breathing and also involuntary breathing (e.g., before or after the experience, or at any moment or period during the journey).

The user's behavior and performance may be tracked over multiple sessions, this history may add valuable data to enhance and predict future experience sessions and may be used to alter or influence the cabin experience in the future. For example, the breathing experience and user performance may be shared across vehicles and with other users; e.g., via web/cloud connected service (e.g., a mobile smart device/phone app or an in-vehicle app).

As an example, an immersive interactive breathing experience based on a multi-phase breathing model for one or more users may be provided. According to a journey-based approach, the system may present and monitor user performance during different exercises, during a single journey and across multiple journeys. A machine learning (ML) and/or artificial intelligence (AI) approach may be used to learn from past experiences, modify and curate future breathing experiences (e.g., multi-phase, symmetric/asymmetric exercises, different GUI themes, cabin-based multi-sensory experiences, etc.).

A multi-sensory experience may engage different senses of the user. A GUI may be provided by a touchscreen, AR/VR, HUD, digital projection, etc., and multi-sensory aspects may incorporate new and existing vehicle functionality such as seating, climatization, ambient lighting, infotainment and audio system, etc.

The system may provide guidance to the user on the breathing behavior/gesture and may compare the sensor data to evaluate the user breathing activity and performance.

The breathing model may be 2-dimensional or 3-dimensional and based on different GUI themes. The IPA and/or avatar may recommend, introduce and guide the user through the experience.

Camera-based sensors may provide real-time breathing data (i.e., for breathing type and performance), user gesture recognition and tracking (enhanced breathing data), and user identification.

A gamified experience driven by user breathing guidance and incorporating a GUI and multi-sensory bio-feedback may be provided. The system may adapt the experience according to the behavior/performance and/or context of the vehicle (e.g., journey length, location in the journey, incoming phone call, etc.) or user (e.g., mood or state of the user (detected or self-reported (e.g., via the GUI))

The disclosure may provide applications to a driver or a passenger, and/or automated, connected, electric and shared (ACES) vehicles (e.g., autonomous level 4 or 5, EV, etc.).

Furthermore, a vehicle comprising the control device described herein and the illustration device (for illustrating the instruction steps of the multi-phase breathing exercise) is provided.

The illustration device may be (or may comprise) a display, e.g., a touchscreen, a center information display, augmented and/or virtual reality glasses, a head-up display.

The vehicle may comprise the sensor device. The sensor device may comprise at least one interior camera (e.g., arranged in the cabin of the vehicle). The sensor device may be configured to record data of the passenger, such as data comprising body movements and/or body gestures of the passenger. The sensor device may be arranged towards a vehicle seat (e.g., on which the passenger is sitting).

The sensor device (e.g., the at least one interior camera) may be configured to record data of the passenger, wherein the data may comprise movement of the passenger's clothing features (e.g., shirt/sweater graphics, buttons, pockets, seams). The movement may be measured by measuring camera pixel movement (e.g., of the clothing features).

The sensor device may comprise multiple sensors and/or multiple sensor types, e.g., an inertial measurement unit, a force and/or strain gauge sensor, a radar, a microphone, an electrocardiogram (ECG) sensor and/or an electroencephalography (EEG) sensor. The sensor device may provide sensor fusion based on data of the multiple sensors and/or sensor types.

The sensor device may comprise at the least one interior camera and the radar, providing sensor fusion based on data of the least one interior camera and the radar.

The vehicle may be a motor vehicle. The vehicle may be a passenger car, especially an automobile, or a commercial vehicle, such as a truck.

The vehicle may be automated. The vehicle may be designed to take over longitudinal guidance and/or lateral guidance by means of the control device during automated driving of the motor vehicle at least partially and/or at least temporarily.

The automated driving may be such that the locomotion of the vehicle is (largely) autonomous. The automated driving can be controlled at least partially and/or at least temporarily by the control device.

It is conceivable that the vehicle may intervene actively, e.g., by adjusting an actual steering wheel position, and optionally passively, e.g., by displaying a turn-off indication, in the transverse guidance of the motor vehicle by a driving assistance system.

The vehicle may be a motor vehicle of autonomy level 0, i.e., the driver assumes the dynamic driving task, even if support systems (e.g., ABS or ESP) are present.

The vehicle may be a motor vehicle of autonomy level 1, i.e., the vehicle may have certain driver assistance systems that support the driver in vehicle operation, such as adaptive cruise control (ACC).

The vehicle can be a motor vehicle of autonomy level 2, i.e., the vehicle may be partially automated in such a way that functions such as automatic parking, lane keeping or lateral guidance, general longitudinal guidance, acceleration and/or braking are performed by driver assistance systems.

The vehicle may be a motor vehicle of autonomy level 3, i.e., the vehicle may be automated on such a conditional basis that the driver does not have to monitor the system vehicle throughout. The vehicle may autonomously perform functions such as triggering the turn signal, changing lanes, and/or lane keeping. The driver may attend to other pursuits, but may prompted by the system to take the lead within a warning time if needed.

The vehicle may be a motor vehicle of autonomy level 4, i.e., the vehicle may be so highly automated that the driving of the vehicle is permanently taken over by the system of the vehicle. If the driving tasks are no longer handled by the system, the driver may be asked to take over the driving.

The vehicle may be a motor vehicle of autonomy level 5, i.e., the vehicle may be fully automated such that the driver is not required to perform the driving task. No human intervention is required except to set the destination and start the system. The vehicle may operate without a steering wheel or pedals.

What is described herein with respect to the control device also applies analogously to the vehicle and vice versa.

Furthermore, a method for assisting a multi-phase breathing exercise of a passenger of a vehicle is provided.

The method comprises controlling an illustration device (e.g., a display) to illustrate instruction steps of the multi-phase breathing exercise to the passenger.

The method comprises monitoring breathing actions and/or states of the passenger while the instructions are being illustrated.

The method comprises evaluating a correlation of the monitored breathing actions and/or states to the illustrated instruction steps.

The method comprises providing a feedback to the passenger based on the evaluated correlation.

The method may be a computer-implemented method, i.e., one, several or all steps of the method may be executed at least partially by a computer or a device for data processing, optionally the control device.

What is described herein with respect to the control device and the vehicle applies analogously to the method and vice versa.

Furthermore, a computer program is provided. A computer program comprises instructions which, when the computer program is executed by a computer, cause the computer to at least partially execute the method described herein.

A program code of the computer program may be in any code, especially in a code suitable for control systems of motor vehicles.

What is described herein with respect to the control device, the vehicle and the method applies analogously to the computer program and vice versa.

Furthermore, a computer-readable medium, in particular a computer-readable storage medium, is provided. The computer-readable medium comprises instructions which, when the instructions are executed by a computer, cause the computer to at least partially execute or perform the method described herein.

Thus, a computer-readable medium comprising a computer program as defined herein may be provided. The computer-readable medium may be any digital data storage device, such as a USB flash drive, a hard disk, a CD-ROM, an SD card, or an SSD card (or SSD drive/SSD hard disk).

The computer program does not necessarily have to be stored on such a computer-readable storage medium in order to be made available to the vehicle, but may also be obtained externally via the Internet or otherwise.

What is described herein with respect to the method, the control device, the computer program and the vehicle also applies analogously to the computer-readable medium and vice versa.

The above-described features and advantages, as well as others, will become more readily apparent to those of ordinary skill in the art by reference to the following detailed description and accompanying drawings.

### BRIEF DESCRIPTION OF THE DRAWINGS

FIG. 1 shows a schematic overview of a vehicle according to an embodiment of the disclosure;

FIG. 2 shows a schematic illustration of a passenger during a multi-phase breathing exercise;

FIG. 3 shows a schematic flow diagram of a method according to an embodiment of the disclosure;

FIG. 4 shows exemplary graphics for symmetric breathing exercises illustrated by an illustration device;

FIG. 5 shows exemplary graphics for asymmetric breathing exercises illustrated by an illustration device;

FIG. 6 shows exemplary graphics for multiple different closed paths representing different symmetric or asymmetric exercises illustrated by an illustration device;

FIG. 7 shows exemplary graphics illustrating the multi-phase breathing exercise using the clock analogy;

FIG. 8 shows the two examples of FIG. 7 with the target at different positions;

FIG. 9 possible representations of the multi-phase breathing exercise illustrated by an illustration device;

FIG. 10 shows an example of graphics of simultaneously illustrating the multi-phase breathing exercise and the evaluated correlation;

FIG. 11 shows another example of graphics of simultaneously illustrating the multi-phase breathing exercise and the evaluated correlation;

FIG. 12 shows a first further schematic illustration of the passenger during the multi-phase breathing exercise; and

FIG. 13 shows a second further schematic illustrations of the passenger during the multi-phase breathing exercise.

### DETAILED DESCRIPTION

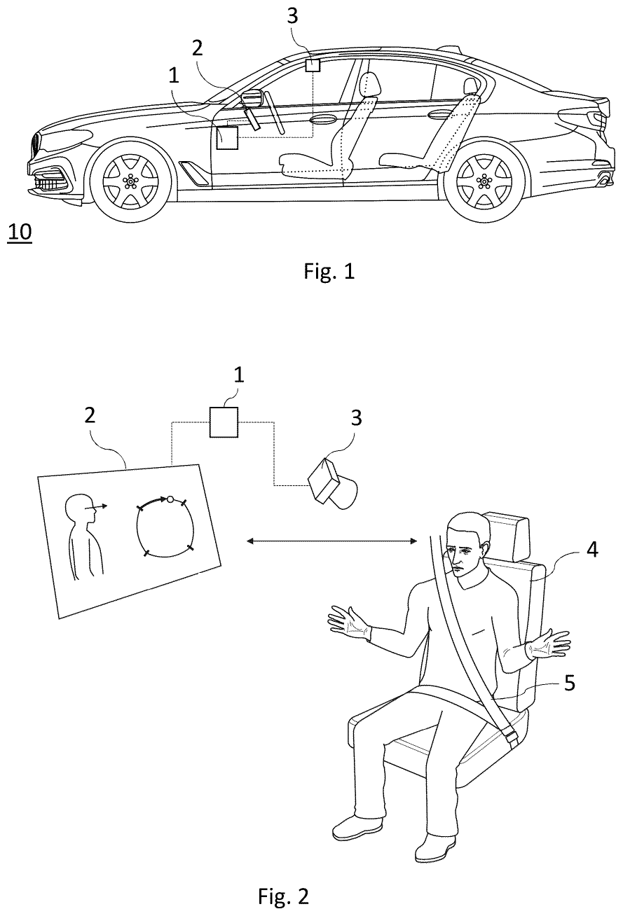

The vehicle 10, which is only schematically shown in FIG. 1, comprises the control device 1, the illustration device 2 (e.g., a display) and a sensor device 3.

The control device 1 and the illustration device 2 are configured to communicate with each other, such that the control device 1 can control the illustration device 2. Additionally, the control device 1 and the sensor device 3 are configured to communicate with each other, such as sending and receiving signals and/or data between both devices.

As illustrated in FIG. 2, the control device 1 is configured to assist a multi-phase breathing exercise of a passenger of the vehicle 10. During such a multi-phase breathing exercise, which may be performed during a journey of the vehicle 10, the passenger is sitting on a vehicle seat 4 in a cabin of the vehicle 10. A seat belt 5 may be fastened, in particular, when the vehicle 10 is driving.

During the multi-phase breathing exercise, the passenger is watching the illustration device 2 which is illustrating instruction steps of the multi-phase breathing exercise. Breathing actions and/or states of the passenger are monitored via the sensor device 3 while the instructions are being illustrated.

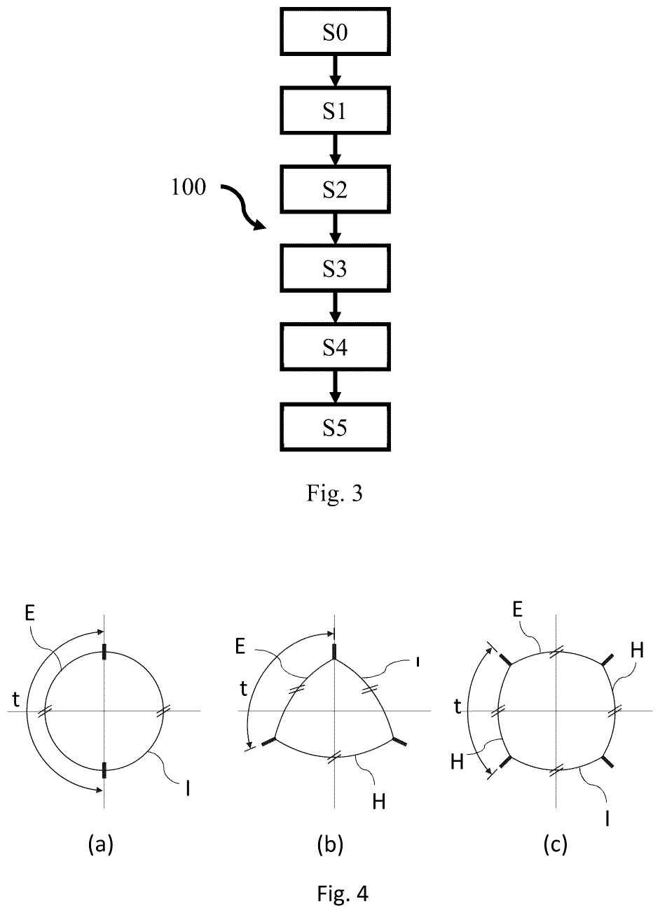

In order to assist the multi-phase breathing exercise of the passenger, the control device 1 is designed to execute the (control) method described below in detail with reference to FIG. 3.

In an optional step S0, the multi-phase breathing exercise may be adapted based on passenger performances in previous breathing exercises, a current journey length, a location in the current journey, a mood of the passenger, and/or a state of the passenger.

In a first step S1, the illustration device 2 is controlled to illustrate the instruction steps of the multi-phase breathing exercise to the passenger.

The multi-phase breathing exercise may be composed of multiple phases comprising at least an inhale phase and at least an exhale phase and optionally at least a breathing hold phase, wherein the illustrated instruction steps specify a breathing duration, a breathing depth and/or a breathing rate for each of the multiple phases.

The instruction steps may comprise additional instructions regarding body gestures, wherein each of the body gestures is a facial and/or hand gesture providing a measure of one of the breathing actions and/or states.

In a second step S2, breathing actions and/or states of the passenger are monitored while the instructions are being illustrated.

For instance, data regarding recordings of at least one chest and/or belly expansion and at least one chest and/or belly contraction of the passenger may be received from the sensor device 3. The at least one chest and/or belly expansion and the at least one chest and/or belly contraction may be then determined in the received data.

If the instruction steps comprise additional instructions regarding body gestures, data regarding recordings of body gestures of the passenger may be received from the sensor device 3, and the body gestures may be then identified in the received data.

In a third step S3, a correlation of the monitored breathing actions and/or states to the illustrated instruction steps is evaluated, e.g., by tracking whether the multiple phases are performed by the passenger in accordance with the illustrated instruction steps.

In a fourth step S4, a feedback is provided to the passenger based on the evaluated correlation.

For example, the feedback may be provided by controlling the illustration device 2 to illustrate the evaluated correlation and/or the monitored breathing actions and/or states simultaneously to the instruction steps in a single representation.

In a fifth step S5, identification data to identify the passenger may be received, and the evaluated correlation, the monitored breathing actions and/or state, and/or results based on the evaluated correlation may be stored in a passenger profile of the identified passenger. The identification of the passenger may be performed prior to the start of the breathing exercise.

FIGS. 4-9 show exemplary graphics how the instruction steps of the multi-phase breathing exercise may be illustrated by the illustration device 2.

The multi-phase breathing exercise may be illustrated as a closed path comprising multiple of the instruction steps which may be repeated by repeating following said closed path. In other words, the illustration may represent time like a clock whilst performing the multi-phase breathing exercise. That way, breathing guidance can be provided by the illustration device 2 by illustrating time in conjunction with the breathing instruction steps for different breathing actions according to the breathing exercise.

The closed path, which may be illustrated as part of a graphical user interface (GUI), may have different shapes according to different types of phase breathing.

FIG. 4 shows exemplary graphics for symmetric breathing exercises. Symmetric refers to the symmetric breathing shape, wherein each part of the closed path representing one of the phases has the same length and shape. The length of each part corresponds to the period of time t (or duration) of the corresponding breathing phase, which is the same for all phases.

The symmetric breathing exercise may be composed of two phases (a) comprising an exhale phase E and an inhale phase I, three phases (b) additionally comprising a breathing hold phase H, or four phases (d) additionally comprising a second breathing hold phase H.

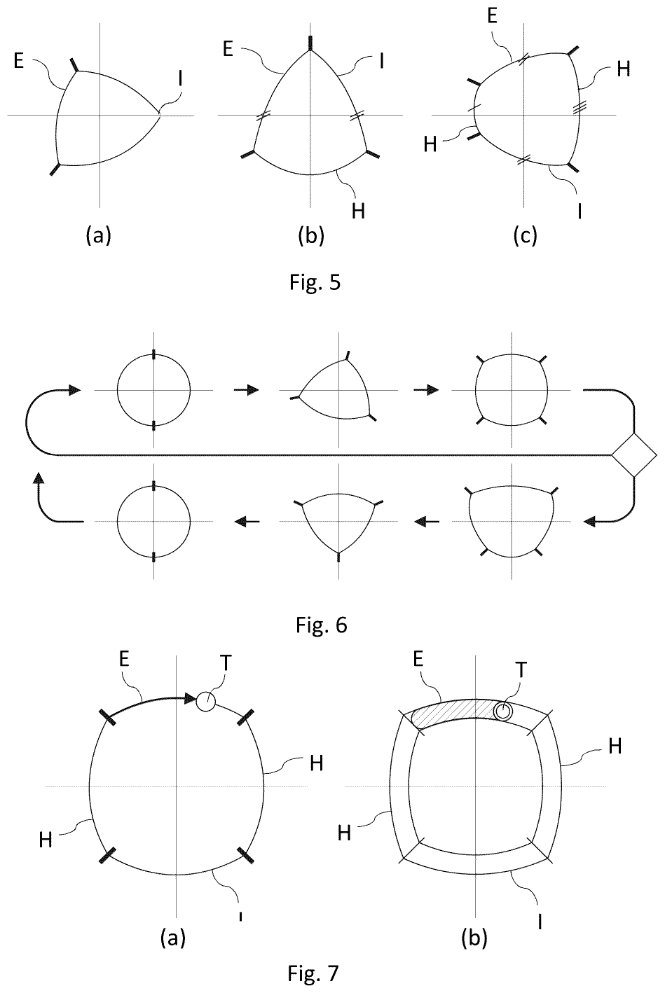

FIG. 5 shows exemplary graphics for asymmetric breathing exercises. Contrary to the symmetric exercises, the different parts of the closed path may have different lengths corresponding to different durations for performing the respective breathing phase.

The asymmetric breathing exercise may be composed of two phases (a), wherein, e.g., the exhale phase E may have a shorter duration than the inhale phase I.

Alternatively, the asymmetric breathing exercise may be composed of three phases (b) or four phases (d), wherein, e.g., the breathing hold phase H (or at least one of the breathing hold phases H) may have a shorter duration than the exhale phase E and the inhale phase I.

These are merely examples for asymmetric breathing, wherein any other lengths, shapes and number of phases are possible. For instance, the exercise may be composed of three phases, wherein each of the phases have different durations, and/or four phases, wherein the exhale phase and the inhale phase may have different durations. Additionally, or alternatively, the closed path may have a simple and/or symmetric (e.g., circular) shape. In this case, the lengths of the different paths of the closed path may be adjusted accordingly to provide the asymmetric breathing exercise.

FIG. 6 illustrates that the multi-phase breathing exercise may comprise multiple different closed paths representing different symmetric or asymmetric exercises which may be illustrated in subsequent order by the illustration device 2, e.g., in the GUI.

The transitions between the different symmetric or asymmetric exercises may be determined and performed at different stages, e.g., based on the type of the passenger, the journey duration, experience and/or expertise of the passenger, and/or a gamification level reached by the passenger. The GUI may be configured to guide the passenger through these transitions.

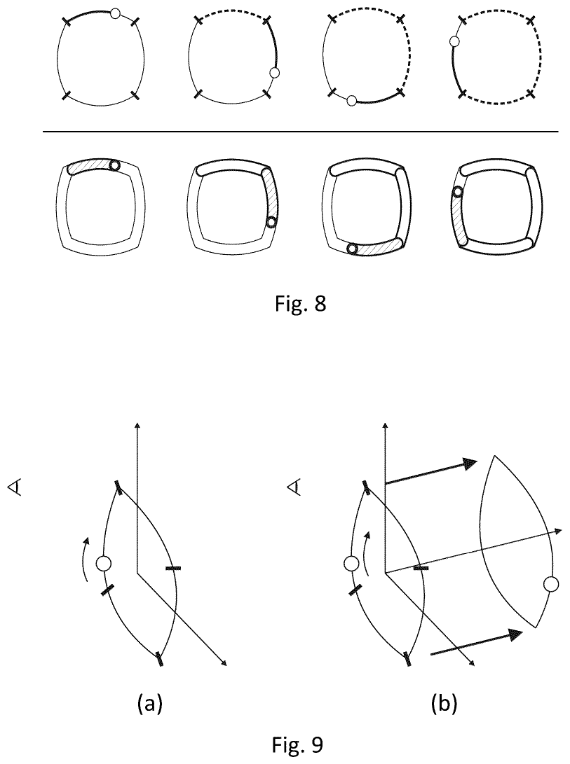

FIG. 7 shows two examples how to illustrate the multi-phase breathing exercise using the clock analogy, i.e., a closed path, wherein the breathing guidance may follow a path (a) or a shape (b). In both examples, the current (phase and time) position to be followed by the passenger is indicated by a, e.g., round, target T. Thus, the target may indicate the breathing process in accordance with the breathing exercise.

FIG. 8 shows the two examples of FIG. 7 with the target T at different positions.

FIG. 9 shows possible representations of the multi-phase breathing exercise within a two-dimensional plane/space (a) and a three-dimensional space (b), which may be simulated or virtual.

The different directions in the plane or space may be used to express and to reinforce different breathing parameters according to the exercise, such as time, phase and/or depth, in order to create a more immersive experience.

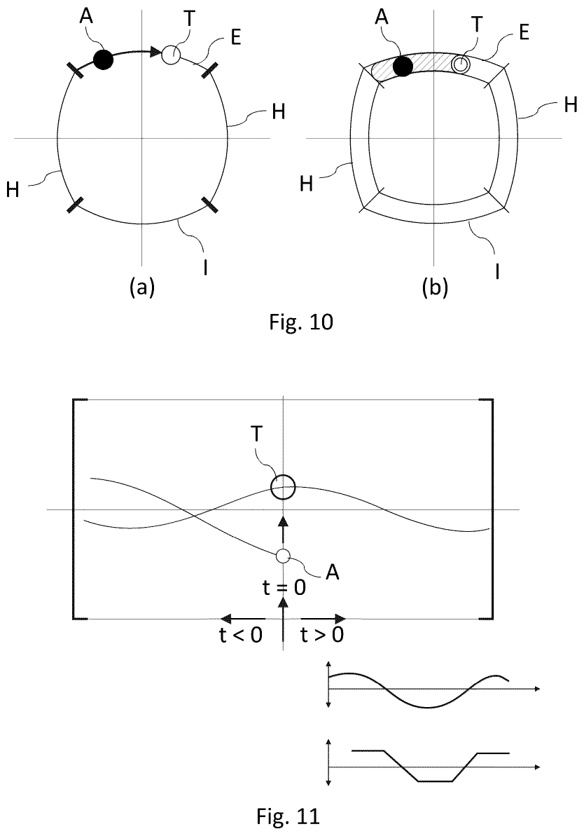

FIG. 10 shows exemplary graphics illustrating the instruction steps of the multi-phase breathing exercise, wherein the respective evaluated correlations with respect to the monitored breathing actions and/or states are illustrated simultaneously in the same graphics. In particular, the breathing guidance follows a path (a) or a shape (b), similar to the graphics shown in FIG. 7.

In addition to the target T, the position of the actual breathing of the passenger based on the monitored breathing actions and/or states is indicated by the marker A. Thus, the passenger can easily identify the correlation and difference between the illustrated exercise and his breathing, and therefore can easily adjust his breathing in accordance with the breathing exercise.

This feedback, which may be also called bio-feedback, allows, e.g., a gamification of the breathing exercise. The gamification may incite the passenger to match the target T, which may be rewarded, e.g., by a scoring.

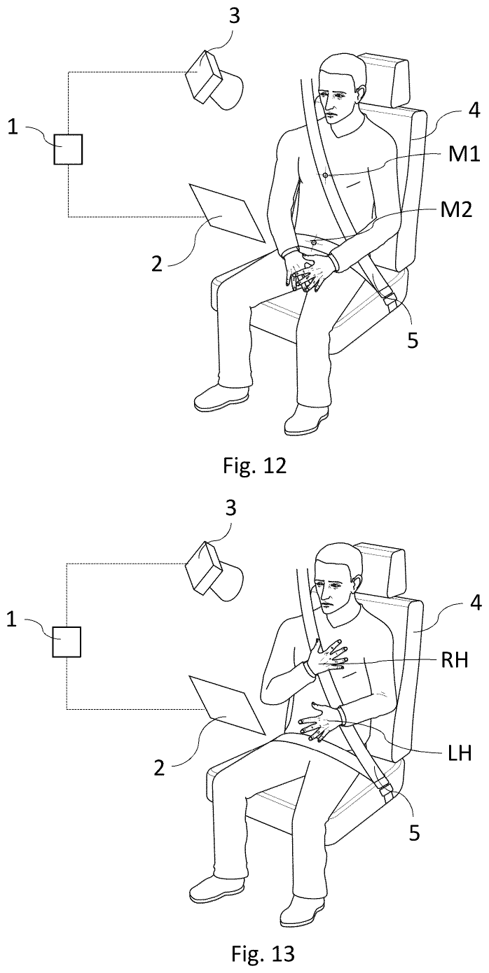

FIG. 11 shows another exemplary graphics illustrating the instruction steps of the multi-phase breathing exercise in conjunction with the evaluated correlation. Instead of illustrating the instruction steps on a closed path based on the clock analogy, the instruction steps and the actual breathing of the passenger are presented by two waves in a coordinate system, wherein the target T and the marker A are indicated at the current time t=0. Thus, the passenger can easily identify the difference between the illustrated exercise and his breathing by comparing the positions of the target T and the marker A. The difference is indicated by the arrow between A and T.

The waves may have different forms such as sinusoidal or a squared form, as shown at the bottom right of FIG. 11.

FIGS. 12 and 13 show further schematic illustrations of the passenger during the multi-phase breathing exercise.

In order to record at least one chest and/or belly expansion and at least one chest and/or belly contraction of the passenger, visual markers M1 and M2 may be arranged on the seat belt 5 at positions close to the passenger's chest and belly, respectively, when the seat belt 5 is fastened, as shown in FIG. 12. These visual markers M1 and M2 help identifying and determining the at least one chest and/or belly expansion and the at least one chest and/or belly contraction in the received data recorded by the sensor device 3.

As an alternative to the markers M1 and M2, the illustrated instructions may comprise instructions to the passenger to place one hand, such as the right hand RH, on the chest and the other hand, such as the left hand LH, on the belly, as shown in FIG. 13. The hands RH, LH may be used as visual markers that can be tracked to directly determine the chest and belly expansion and contraction that directly correlates with the passenger's breathing.

The visual markers M1 and M2 may provide general information on whether the passenger is following the breathing guidance and/or instructions and/or whether the passenger is engaged in the exercise (e.g., if the exercise is interrupted or the passenger stops, e.g., if the passenger receives a phone call). The exercise may be paused or stopped until the passenger re-establishes the visual markers M1 and M2 (e.g., hands in position).

### LIST OF REFERENCE SIGNS

1 control device
2 illustration device
3 sensor device
4 vehicle seat
5 seat belt
10 vehicle
E exhale phase
I inhale phase
H hold breathing phase
t time
T target
A marker of monitored breathing actions and/or states
M1, M2 markers on seat belt
LH, RH left and right hand of passenger
100 method
S0-S5 method steps

1. A control device for assisting a multi-phase breathing exercise of a passenger of a vehicle, the control device configured to:
control an illustration device to illustrate instruction steps of the multi-phase breathing exercise to the passenger;
monitor aspects of the passenger while the instructions are being illustrated;
evaluate a correlation of the monitored aspects to the illustrated instruction steps; and
provide a feedback to the passenger based on the evaluated correlation;
wherein the monitored aspects of the passenger include at least one of the group consisting of breathing actions of the passenger and states of the passenger.

2. The control device according to claim 1, wherein:
the multi-phase breathing exercise is composed of multiple phases comprising at least an inhale phase and at least an exhale phase and optionally at least a breathing hold phase, wherein the illustrated instruction steps specify at least one of the group consisting of a breathing duration, a breathing depth and a breathing rate for each of the multiple phases; and
the control device is configured to evaluate the correlation by tracking whether the multiple phases are performed by the passenger in accordance with the illustrated instruction steps.

3. The control device according to claim 2, wherein the control device is configured to:
receive identification data regarding the passenger; and
store in a passenger profile associated with the identification data, at least one of the group consisting of the evaluated correlation, the aspects of the passenger, and results based on the evaluated correlation.

4. The control device according to claim 2, wherein:
the instruction steps comprise additional instructions regarding body gestures, each of the body gestures providing a measure of one of the aspects of the passenger; and
the control device is configured to monitor the aspects of the passenger by:
receiving, from a sensor device, data regarding recordings of body gestures of the passenger; and
identifying the body gestures in the received data.

5. The control device according to claim 4, wherein the body gesture is at least one of the group consisting of a facial gesture and a hand gesture.

6. The control device according to claim 1, wherein:
the instruction steps comprise additional instructions regarding at least one body gesture, each of the at least one body gestures providing a measure of one of the aspects of the passenter; and
the control device is configured to monitor the aspects of the passenger by:
receiving, from a sensor device, data regarding recordings of the at least one body gesture of the passenger; and
identifying the at least one body gesture in the received data.

7. The control device according to claim 6, wherein the at least one body gesture includes at least one of the group consisting of a facial gesture and hand gesture.

8. The control device according to claim 7, wherein the at least one body gesture includes a facial gesture.

9. The control device according to claim 6, wherein the control device is configured to monitor the aspects of the passenger by:
receiving, from a sensor device, data regarding recordings of at least one bodily expansion and at least one bodily contraction of the passenger, wherein the bodily expansion is at least one of the group consisting of a chest expansion and a belly expansion; and
determining the at least one bodily expansion and the at least one bodily contraction in the received data.

10. The control device according to claim 1, wherein the control device is configured to monitor the aspects of the passenger by:
receiving, from a sensor device, data regarding recordings of at least one bodily expansion and at least one bodily contraction of the passenger, wherein the bodily expansion is at least one of the group consisting of a chest expansion and a belly expansion; and
determining the at least one bodily expansion and the at least one bodily contraction in the received data.

11. The control device according to claim 1, wherein the control device is configured to provide the feedback by:
controlling the illustration device to illustrate, simultaneously to the instruction steps in a single representation, at least one of the group consisting of: the evaluated correlation, the monitored aspects of the passenger.

12. The control device according to claim 1, wherein the control device is configured to:
receive identification data regarding the passenger; and
store in a passenger profile associated with the identification data, at least one of the group consisting of the evaluated correlation, the aspects of the passenger, and results based on the evaluated correlation.

13. The control device according to claim 12, wherein the control device is configured to:
adapt the multi-phase breathing exercise based on passenger performances in previous breathing exercises.

14. The control device according to claim 1, wherein the control device is configured to:
adapt the multi-phase breathing exercise based on at least one of the group consisting of passenger performances in previous breathing exercises, a current journey length, a location in the current journey, a mood of the passenger, and a state of the passenger.

15. A vehicle comprising:
the control device according to claim 1,
the illustration device for illustrating the instruction steps of the multi-phase breathing exercise.

16. A method for assisting a multi-phase breathing exercise of a passenger of a vehicle, the method comprising:
controlling an illustration device to illustrate instruction steps of the multi-phase breathing exercise to the passenger;
monitoring aspects of the passenger while the instructions are being illustrated;
evaluating a correlation of the monitored aspects of the passenger to the illustrated instruction steps; and
providing feedback to the passenger based on the evaluated correlation
wherein the monitored aspects of the passenger include at least one of the group consisting of breathing actions of the passenger and states of the passenger.

17. A non-transitory computer storage medium comprising instructions which, when executed by a computer, cause the computer to carry out the method according to claim 16.
